(19) United States
(12) Patent Application Publication
(10) Pub. No.: US 2011/0075910 A1
(43) Pub. Date: Mar. 31, 2011

(54) X-ray imaging system, imaging method and computer readable media including imaging program

(75) Inventors: Eiichi Kanagawa, Kanagawa (JP); Noriaki Ida, Kanagawa (JP); Sadato Akahori, Kanagawa (JP); Yasunori Ohta, Kanagawa (JP)

(73) Assignee: FUJIFILM Corporation, Tokyo (JP)

(21) Appl. No.: 12/923,611

(22) Filed: Sep. 29, 2010

(30) Foreign Application Priority Data
Sep. 30, 2009 (JP) 2009-226919

(51) Int. Cl. G06K 9/00; A61B 6/03

(52) U.S. Cl. 382/131; 378/4

(57) ABSTRACT

An X-ray imaging system includes an imager in which a subject is irradiated with X-ray at different angles while moving an X-ray source in one direction and the X-ray with which the subject has been irradiated is detected with a flat panel detector to acquire projection data of X-ray images taken at the different angles in tomosynthesis imaging, and an image processor in which an X-ray tomographic image is reconstructed not by using abnormal pixel data corresponding to defective pixels but by using only pixel data corresponding to normal pixels from among pixel data making up the projection data of the X-ray images acquired by the imager by reference to a defective pixel map in which information on the defective pixels due to the flat panel detector and its peripheral circuit has been stored.

### BACKGROUND OF THE INVENTION

The present invention relates to an X-ray imaging system, an imaging method, and an imaging program for reconstructing an X-ray tomographic image at a cross section located at a given height of a subject using projection data of X-ray images acquired by tomosynthesis imaging.

An X-ray imaging system for tomosynthesis imaging irradiates a subject with X-ray at different angles while moving an X-ray source in one direction and detects the X-ray with which the subject has been irradiated with a flat panel X-ray detector (FPD) to achieve acquisition of projection data corresponding to X-ray images of the subject taken at the different angles by a single imaging operation. Then the process proceeds to image processing using the projection data of the acquired X-ray images to reconstruct an X-ray tomographic image at a cross section of the subject at a given height thereof.

Now, reconstruction of an X-ray tomographic image will be described.

In tomosynthesis imaging, an X-ray source is moved in one direction and a subject 30 is irradiated with X-ray from positions S1, S2, and S3 to obtain X-ray images (projection data) P1, P2, and P3 of the subject 30 as illustrated in FIG. 5A.

Now considering the case where imaging objects A and B are present at two positions different in height of the subject 30 as illustrated in FIG. 5A, the X-ray emitted from the imaging positions S1, S2 and S3 by the X-ray source passes through the subject 30 to enter an FPD. As a result, the two imaging objects A, B are projected at different positions on the X-ray images P1, P2 and P3 corresponding to the imaging positions S1, S2 and S3.

In the X-ray image P1, for example, the X-ray source, located in the position S1 to the left of the imaging objects A, B with respect to the direction of movement of the X-ray source, causes projections of the imaging objects A, B to be formed in positions P1A, P1B that are set off to the right of the imaging objects A, B. Likewise, in the X-ray image P2, the projections are formed in positions P2A, P2B that are substantially directly beneath the imaging objects A, B; in the X-ray image P3, the projections are formed in positions P3A, P3B that are set off to the left of the imaging objects A, B.

To reconstruct an X-ray tomographic image of the subject at a cross section located at the height of the imaging object A, the X-ray image P1 is shifted leftward, and the X-ray image P3 is shifted rightward, for example, so that the projection positions P1A, P2A, and P3A of the imaging object A coincide with each other as illustrated in FIG. 5B (shift addition method). Thus, an X-ray tomographic image is reconstructed wherein the cross section located at the height of the imaging object A is accentuated. An X-ray tomographic image at a cross section located at a given height containing a cross section at the height of the imaging object B may likewise be reconstructed.

The FPD comprises photoelectric conversion elements corresponding to the respective pixels of an X-ray image arranged in matrix form. However, there are a pixel at which X-ray cannot be detected, and a pixel having a different X-ray detection sensitivity from other pixels mainly because of problems in manufacturing technology and the peripheral circuit. Such a pixel of an image produced from the pixel of the FPD is hereinafter referred to as “defective pixel”. In order to reduce or eliminate adverse effects of the defective pixels on the image, the defective pixels on the FPD panel are kept in mind beforehand and various corrections are performed on the projection data read out of the FPD.

JP 2007-632 A, for example, relates to compensation of an offset signal produced by a flat panel detector of a radiographic imaging apparatus. The literature describes an offset compensation for the flat panel detector using an offset map.

### SUMMARY OF THE INVENTION

As described above, however, tomosynthesis imaging acquires, during a single imaging operation, projection data corresponding to a plurality of X-ray images, each of which has a large size, resulting in projection data having a large data quantity. Accordingly, where each and every image data having a large data quantity such as projection data of X-ray images as acquired in tomosynthesis imaging was corrected as proposed in JP 2007-632 A, reconstruction of an X-ray tomographic image required a long time.

An object of the present invention is to solve the above problems associated with the prior art and provide an X-ray imaging system, an imaging method and an imaging program capable of reconstructing an X-ray tomographic image by tomosynthesis imaging at a high speed within a short time period.

In order to achieve the above object, the present invention provides an X-ray imaging system comprising:

an imager in which a subject is irradiated with X-ray at different angles while moving an X-ray source in one direction and the X-ray with which said subject has been irradiated is detected with a flat panel detector to acquire projection data of X-ray images taken at the different angles in tomosynthesis imaging; and

an image processor in which an X-ray tomographic image is reconstructed not by using abnormal pixel data corresponding to defective pixels but by using only pixel data corresponding to normal pixels from among pixel data making up the projection data of the X-ray images acquired by said imager by reference to a defective pixel map in which information on the defective pixels due to said flat panel detector and its peripheral circuit has been stored.

The present invention also provides an X-ray imaging method comprising the steps of:

irradiating a subject with X-ray at different angles while moving an X-ray source in one direction, and detecting the X-ray with which said subject has been irradiated with a flat panel detector to acquire projection data of X-ray images taken at the different angles in tomosynthesis imaging; and

reconstructing an X-ray tomographic image not by using abnormal pixel data corresponding to defective pixels but by using only pixel data corresponding to normal pixels from among pixel data making up the projection data of the X-ray images by reference to a defective pixel map in which information on the defective pixels due to said flat panel detector and its peripheral circuit has been stored.

The present invention also provides a computer readable media including an X-ray imaging program for causing a computer to execute:

a step of receiving projection data of X-ray images taken at different angles acquired by irradiating a subject with X-ray at the different angles while moving an X-ray source in one direction and detecting the X-ray with which said subject has been irradiated with a flat panel detector in tomosynthesis imaging;

a step of identifying abnormal pixel data corresponding to defective pixels of said flat panel detector from among pixel data making up the received projection data of the X-ray images by reference to a defective pixel map in which information on the defective pixels due to said flat panel detector and its peripheral circuit has been stored;

a step of performing a first correction for masking the identified abnormal pixel data corresponding to the defective pixel; and

a step of reconstructing a first X-ray tomographic image by using the projection data of the X-ray images after said first correction.

The present invention reconstructs an X-ray tomographic image not by using pixel data of an X-ray image corresponding to defective pixels of a flat panel detector but by using only pixel data corresponding to normal pixels, whereby the X-ray tomographic image can be reconstructed at a high speed within a short time period.

### BRIEF DESCRIPTION OF THE DRAWINGS

FIG. 1 is a block diagram representing a configuration of an X-ray imaging system according to an embodiment of the invention.

FIG. 2 is a block diagram illustrating a detailed configuration of an image processor in the imaging system shown in FIG. 1.

FIG. 3 is a conceptual view illustrating a positional relationship between an X-ray source, a subject and a defective pixel of an FPD in tomosynthesis imaging.

FIG. 4 is a conceptual view illustrating a positional relationship between the X-ray source, an imaging object of the subject and the defective pixel of the FPD in the imaging system shown in FIG. 1.

FIGS. 5A and 5B are conceptual views illustrating reconstruction of an X-ray tomographic image in tomosynthesis imaging.

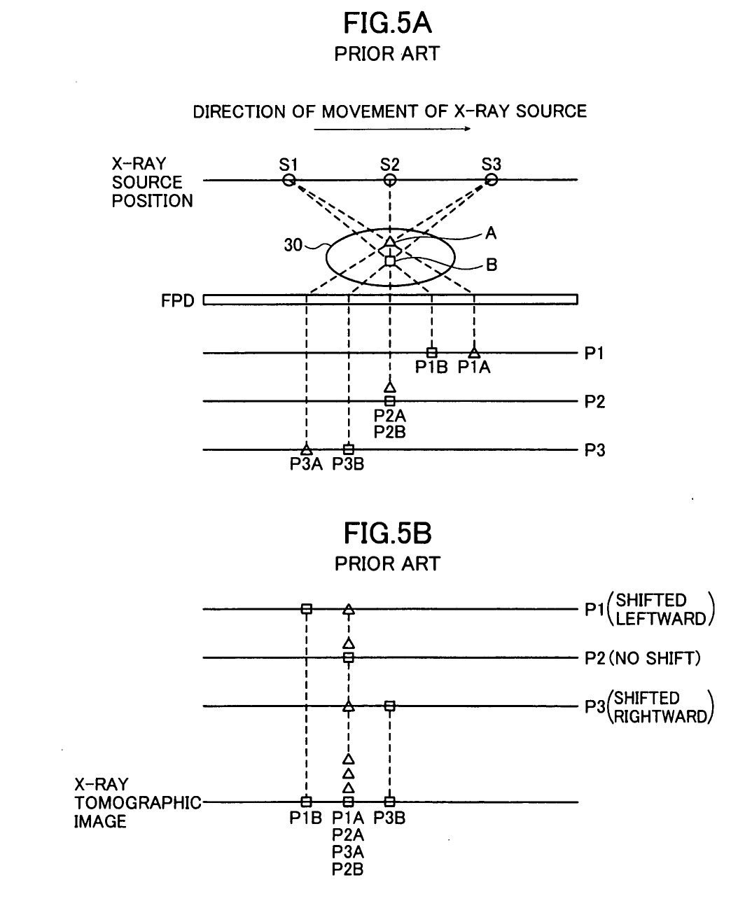

### DETAILED DESCRIPTION OF THE INVENTION

The X-ray imaging system, imaging method, and imaging program of the invention will be described in detail with reference to the preferred embodiments shown in the accompanying drawings.

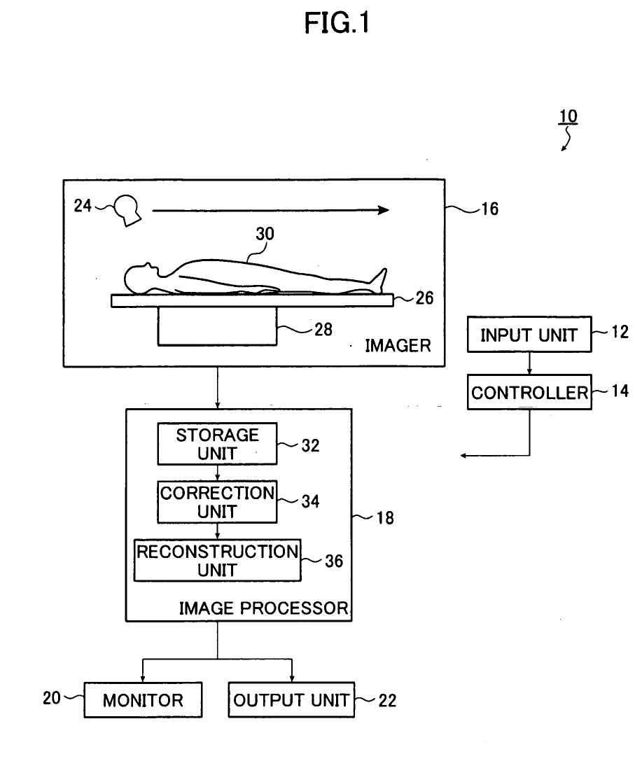

FIG. 1 is a block diagram representing a configuration of an X-ray imaging system according to an embodiment of the invention. An X-ray imaging system 10 shown in FIG. 1 acquires images of a subject 30 such as a human body by tomosynthesis imaging (X-ray imaging) and reconstructs an X-ray tomographic image at a cross section located at a given height of the subject 30. The imaging system 10 comprises an input unit 12, a controller 14, an imager 16, an image processor 18, a monitor 20, and an output unit 22.

The input unit 12 is provided to enter various instructions including an instruction to start imaging and an instruction to switch operations described later and may include a mouse, a keyboard, etc. The input unit 12 produces an instruction signal, which is received by the controller 14.

The controller 14 produces control signals according to an instruction signal transmitted from the input unit 12 to control the operations of the imaging system 10 including imaging operations by the imager 16, image processing by the image processor 18, screen display by the monitor 20, and output processing by the output unit 22. Although not shown, the control signals outputted from the controller 14 are received by the imager 16, the image processor 18, the monitor 20 and the output unit 22.

The imager 16 acquires images of the subject 30 by tomosynthesis imaging according to a control signal supplied from the controller 14; it comprises an X-ray source 24, a carrier (not shown) for moving the X-ray source 24, an imaging table 26, and a flat panel type X-ray detector (FPD) 28.

The X-ray source 24 is spaced apart by a predetermined distance above the subject 30 that is disposed on the top surface of the imaging table 26.

The FPD 28 is disposed on the bottom side of the imaging table 26 so that its X-ray receiving surface faces upwards. The FPD 28 detects X-ray having passed through the subject 30 and effects photoelectric conversion to produce digital image data (projection data) corresponding to an acquired X-ray image of the subject 30. The FPD 28 may be of a direct type in which radiation is directly converted into an electric charge, an indirect type in which radiation is temporarily converted into light, which is then converted into an electric signal, or any of various other types. The FPD 28 may be configured so that it is movable in the same direction as the X-ray source 24.

In tomosynthesis imaging, the carrier controls the movement of the X-ray source 24 to move the X-ray source 24 in one direction and change the X-ray irradiation angle toward the subject 30 so as to irradiate the subject 30 with X-ray at different imaging angles (i.e., at given time intervals). The X-ray emitted by the X-ray source 24 passes through the subject 30 to enter the light receiving surface of the FPD 28, and the FPD 28 detects the X-ray and converts the detected X-ray into electricity to obtain projection data corresponding to the acquired X-ray. image of the subject 30.

In tomosynthesis imaging, a plurality of X-ray images, for example, 20 to 80 images, of the subject 30 having at different imaging angles are taken by a single imaging operation, whereupon the FPD 28 sequentially outputs projection data corresponding to the acquired X-ray images.

Subsequently, the image processor 18 receives the projection data of the X-ray images acquired by the imager 16 according to the control signal supplied from the controller 14 and performs image processing (including correction and image synthesis) using the projection data of the X-ray images to reconstruct an X-ray tomographic image of the subject 30 at a cross section thereof located at a given height. The image processor 18 comprises a storage unit 32, a correction unit 34, and a reconstruction unit 36.

The storage unit 32 stores the projection data of the X-ray images acquired by the imager 16. The correction unit 34 performs a given correction to the projection data of the X-ray images stored in the storage unit 32. The reconstruction unit 36 uses the projection data of the X-ray images corrected by the correction unit 34 to perform image synthesis, thus reconstructing an X-ray tomographic image of the subject 30 at a cross section thereof at a given height. The image processor 18 will be described later in detail.

The image processor 18 may be configured by hardware (a device) or a program for causing a computer to execute a part of the X-ray image processing method of the invention.

Subsequently, the monitor 20 displays an X-ray tomographic image reconstructed by the image processor 18 according to the control signal supplied from the controller 14 and may be exemplified by a flat panel display such as a liquid crystal display.

The output unit 22 outputs an X-ray tomographic image reconstructed by the image processor 18 according to the control signal supplied from the controller 14 and may be exemplified by a thermal printer for printing out the X-ray tomographic image and a storage device capable of storing digital image data of the X-ray tomographic image in any of various recording media.

Next, the image processor 18 will be described in detail.

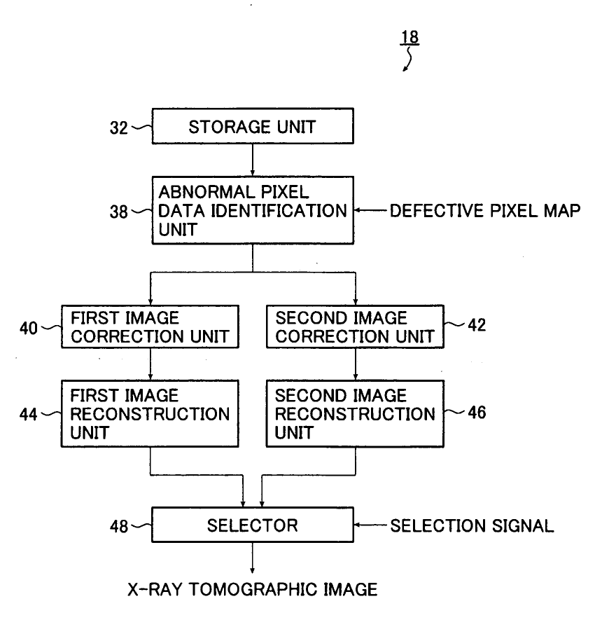

FIG. 2 is a block diagram illustrating a detailed configuration of the image processor shown in FIG. 1. As shown in FIG. 2, the image processor 18 comprises the storage unit 32, an abnormal pixel data identification unit 38, first and second image correction units 40, 42, first and second image reconstruction units 44, 46, and a selector 48. The abnormal pixel data identification unit 38 and the first and second image correction units 40, 42 correspond to the correction unit 34 and the first and second image reconstruction units 44, 46, and the selector 48 correspond to the reconstruction unit 36.

From among the pixel data of the projection data of the X-ray images taken at different imaging angles by the imager 16 and stored in the storage unit 32, the abnormal pixel data identification unit 38 identifies abnormal pixel data corresponding to defective pixels due to the FPD 28 and its peripheral circuit by reference to a defective pixel map.

Information on defective pixels due to the FPD 28 and its peripheral circuit (including positional information and information on the state of the defective pixels) is stored in the defective pixel map. The defective pixel map may be produced, for example, based on projection data of an X-ray image taken by an X-ray imaging in the absence of the subject 30. The defective pixel map is stored in, for example, the FPD 28 or the controller 14.

The defective pixel is a pixel incapable of outputting pixel data proportional to the received X-ray dosage such as a normal pixel, and it includes a pixel at which X-ray cannot be detected because of, for example, a problem in manufacturing technology and a pixel having a different X-ray detection sensitivity from other normal pixels. One defective pixel map may be used or a plurality of different defective pixel maps may be used according to the state of the defective pixels such as the pixel defect and faulty gain to perform corrections to be described later.

Subsequently, the first image correction unit 40 performs a correction for masking the abnormal pixel data (pixel values) corresponding to the defective pixels identified by the abnormal pixel data identification unit 38. There is no particular limitation on the method of masking the abnormal pixel data corresponding to the defective pixels, and the abnormal pixel data corresponding to the defective pixels can be masked by setting the abnormal pixel data corresponding to the defective pixels at 0 assuming that the pixel data ranges from 0 to n where 0 represents the minimum density (transparent) and n represents the maximum density.

The first image reconstruction unit 44 uses the projection data of the X-ray images corrected by the first correction unit 40 to perform image synthesis, thus reconstructing a first X-ray tomographic image. In other words, the first image reconstruction unit 44 uses not the abnormal pixel data corresponding to the defective pixels but only the pixel data corresponding to the normal pixels to reconstruct a first X-ray tomographic image at a cross section located at a given height of the subject 30.

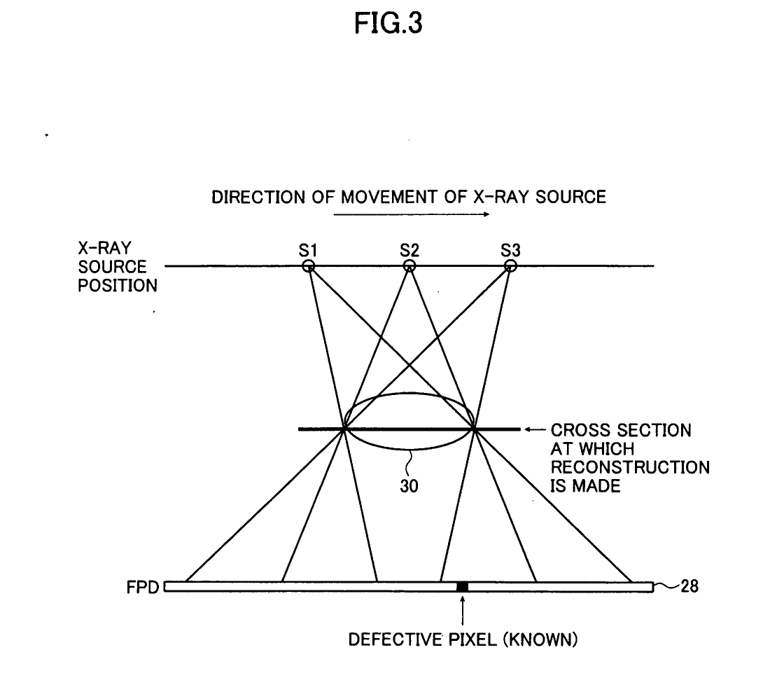

As shown in FIG. 3, in tomosynthesis imaging, each of a plurality of X-ray images acquired by a single imaging operation has a different imaging angle, whereupon the positional relationship between the subject 30 and the defective pixels on the FPD 28 is different (shifted) from one X-ray image to another. Therefore, the, X-ray tomographic image at a cross section of the subject at a given height can be reconstructed by using not the abnormal pixel data corresponding to the defective pixels but the pixel data corresponding to the normal pixels in the projection data of other X-ray images.

FIG. 3 is a conceptual view illustrating a positional relationship between the X-ray source, the subject and the defective pixel of the FPD in tomosynthesis imaging. It is assumed that a defective pixel known from the defective pixel map is present on the FPD 28 at a position on the right side from the subject 30 with respect to the direction of movement of the X-ray source as shown in FIG. 3. In tomosynthesis imaging, the X-ray source is moved in one direction to irradiate the subject 30 with X-ray from positions S1, S2 and S3.

As is seen from FIG. 3, when the subject 30 is irradiated with X-ray from the imaging position S1, the defective pixel on the FPD 28 is located on the left side from the center of the X-ray image of the subject, and when the subject 30 is irradiated with X-ray from the imaging position S2, the defective pixel is projected on the right side from the center of the X-ray image of the subject 30. When the subject 30 is irradiated with X-ray from the imaging position S3, the defective pixel is not included in the X-ray image of the subject 30.

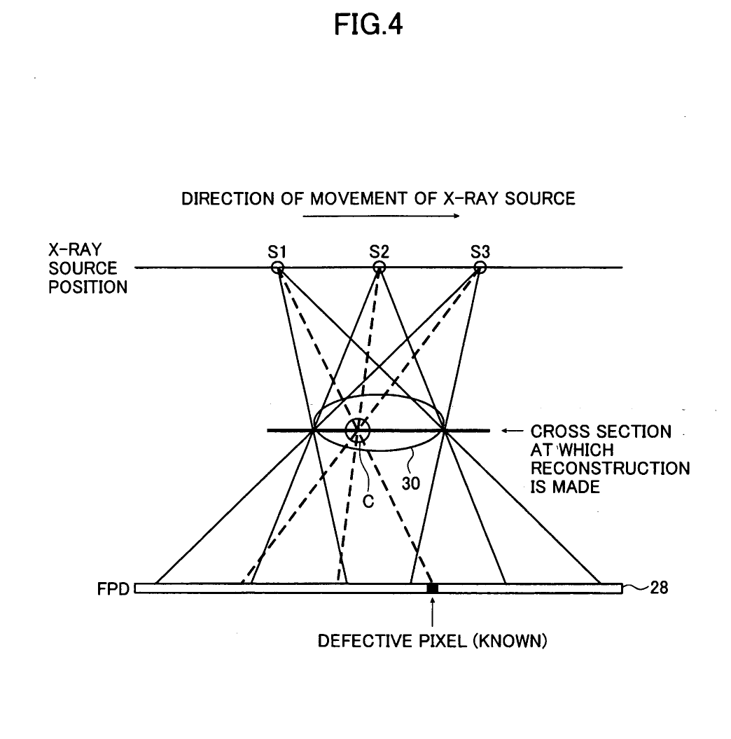

FIG. 4 is a conceptual view illustrating a positional relationship between the X-ray source, the imaging object of the subject and the defective pixel of the FPD in the imaging system shown in FIG. 1. The imaging positions S1, S2, S3 and the position of the defective pixel on the FPD 28 are the same as shown in FIG. 3. In FIG. 4, the imaging object C is located on the left side from the center of the subject 30 with respect to the direction of movement of the X-ray source, and when the subject 30 is irradiated with X-ray from the imaging position S1, the X-ray image of the imaging object C is projected on the defective pixel of the FPD 28.

When an X-ray tomographic image of the subject at a cross section located at the height of the imaging object C is reconstructed in the imaging system 10, from among the pixel data of the X-ray image of the imaging object C acquired at the imaging position S1, the abnormal pixel data corresponding to the defective pixel on the FPD 28 is not used (is masked) but the pixel data corresponding to the normal pixels in the X-ray images of the imaging object C acquired at the other imaging positions S2, S3 are only used to reconstruct the X-ray tomographic image in which the cross section located at the height of the imaging object C is accentuated.

On the other hand, the second image correction unit 42 corrects the abnormal pixel data corresponding to the defective pixel identified by the abnormal pixel data identification unit 38 for each of the X-ray images. The abnormal pixel data may be corrected by a method in which pixel data of normal pixels on the periphery of an abnormal pixel on the same X-ray image (e.g., 8 neighboring pixels surrounding the abnormal pixel) are used to correct the abnormal pixel data by, for example, substitution or interpolation, and a method in which the projection data of other X-ray images acquired by taking at different angles are utilized and pixel data of the normal pixel in the other X-ray images corresponding to the abnormal pixel and the surrounding normal pixels are used to correct the abnormal pixel data by, for example, substitution or interpolation. Such corrections may be implemented using various methods including known methods.

The second image reconstruction unit 46 uses the projection data of the X-ray images corrected by the second image correction unit 42 to perform image synthesis, thus reconstructing a second X-ray tomographic image. In other words, the second image reconstruction unit 46 uses the pixel data corresponding to the normal pixels and the abnormal pixel data corresponding to the corrected defective pixels in the respective X-ray images to reconstruct a second X-ray tomographic image at the cross section of the subject 30 at the same height as in the first image reconstruction unit 44.

The first image correction unit 40 and the second image correction unit 42 perform various corrections including offset correction, residual image correction, gain correction, defective pixel correction, step correction, longitudinal inconsistent density correction, and lateral inconsistent density correction for each of the X-ray images. The offset correction, residual image correction, gain correction, defective pixel correction, step correction, longitudinal inconsistent density correction, and lateral inconsistent density correction are known corrections and may be implemented each using any of various methods including known methods.

The first X-ray tomographic image is reconstructed without using the abnormal pixel data corresponding to the defective pixels on the FPD 28 and is therefore inferior in image quality to the second X-ray tomographic image but can be reconstructed at a higher speed in a shorter time period. On the other hand, the second X-ray tomographic image is reconstructed after correction of the abnormal pixel data corresponding to the defective pixels on the FPD 28 and therefore requires more time for the reconstruction than the first X-ray tomographic image but is of a higher quality.

Subsequently, the selector 48 outputs an X-ray tomographic image as it switches between the first X-ray tomographic image and the second X-ray tomographic image at a given timing according to the selection signal (control signal) supplied from the controller 14.

The second image correction unit 42, the second image reconstruction unit 46, and the selector 48 are not essential components of the image processor 18. These components are preferably provided as necessary when the output of the second X-ray tomographic image is required.

The timing at which selection is made in the selector 48 between the first X-ray tomographic image and the second X-ray tomographic image according to the selection signal is not limited in any manner, permitting use of various timings.

For example, the timing may be so set that the first X-ray tomographic image is outputted from the selector 48 after the first image reconstruction unit 44 has completed reconstruction of the first X-ray tomographic image and the second X-ray tomographic image is outputted from the selector 48 after the second image reconstruction unit 46 has completed reconstruction of the second X-ray tomographic image.- According to this method, the first X-ray tomographic image is first displayed on the monitor 20 at a high speed in a short time period, then the second X-ray tomographic image having a higher image quality is automatically (unconditionally) displayed on the monitor 20.

Alternatively, when a given time period has elapsed from the completion of reconstruction of the second X-ray tomographic image, a switch may be made from the first X-ray tomographic image to the second X-ray tomographic image to output the selected X-ray tomographic image from the selector 48. According to this method, when two or more first X-ray tomographic images are successively displayed in a shorter time than a given time period, the second X-ray tomographic image is not displayed. On the other hand, when the user, interested, allows the first X-ray tomographic image to be displayed longer than a given time period, the second X-ray tomographic image is automatically displayed.

Alternatively, after the completion of reconstruction of the second X-ray tomographic image, a switch may be made between the first X-ray tomographic image and the second X-ray tomographic image according to an instruction (switching instruction) given from the outside through the input unit 12 to output the selected X-ray tomographic image from the selector 48. According to this method, the user is allowed to selectively display the first X-ray tomographic image or the second X-ray tomographic image at any timing desired for any X-ray tomographic image.

After the completion of reconstruction of the first X-ray tomographic image in the first image reconstruction unit 44, the first X-ray tomographic image is outputted from the selector 48 and for the portions (pixels) in which abnormal pixel data was not used to reconstruct the first X-ray tomographic image, the abnormal pixel data portions in the first X-ray tomographic image are sequentially replaced in real time by the corresponding abnormal pixel data portions in the reconstructed second X-ray tomographic image (image data portion of the second X-ray tomographic image corresponding to the abnormal pixel data of the first X-ray tomographic image) each time one abnormal pixel data portion of the second X-ray tomographic image is reconstructed in the second image reconstruction unit 46 until reconstruction of the second X-ray tomographic image is completed. According to this method, a switch is made from the first X-ray tomographic image to the second X-ray tomographic image for a given unit (the unit may be a pixel, a line of pixels, etc.) during reconstruction of the second X-ray tomographic image in lieu of making a switch from the first X-ray tomographic image to the second X-ray tomographic image after the completion of reconstruction of whole of the second X-ray tomographic image. Thus, a high-definition X-ray tomographic image can be displayed by unit in order of reconstruction.

The second image reconstruction unit 46 may be configured such that the abnormal pixel data corresponding to the defective pixels identified by the abnormal pixel data identification unit 38 is used for each of the X-ray images to reconstruct only the pixels corresponding to the defective pixels in the second X-ray tomographic image. In this way, the second image reconstruction unit 46 reconstructs only the pixels corresponding to the defective pixels on the FPD 28, thus enabling the reconstruction at a higher speed.

Next, the operation of the imaging system 10 in tomosynthesis imaging will be described.

The subject 30 is positioned on the top surface of the imaging table 26, whereupon the input unit 12 gives instruction to start imaging, thereby starting tomosynthesis imaging controlled by the controller 14.

Upon start of imaging, the imager 16 irradiates the subject 30 with X-ray as the carrier moves the X-ray source 24 in one direction and changes the X-ray irradiation angle of the X-ray source 24 with respect to the subject 30 so that the subject 30 may be irradiated with X-ray at different imaging angles to acquire X-ray images taken at the different imaging angles during a single imaging operation. Each time an X-ray image of the subject 30 is acquired, the FPD 28 produces projection data corresponding to the X-ray image acquired.

The storage unit 32 of the image processor 18 stores the projection data of the X-ray images acquired by the imager 16.

Subsequently, from among the pixel data that makes up the projection data of the X-ray images stored in the storage unit 32, the abnormal pixel data identification unit 38 identifies the abnormal pixel data corresponding to the defective pixels on the FPD 28 by reference to the defective pixel map.

Then, the first image correction unit 40 performs a correction for masking the identified abnormal pixel data corresponding to the defective pixels; the first image reconstruction unit 44 uses the projection data of the X-ray images corrected by the first image correction unit 40 to reconstruct the first X-ray tomographic image at a cross section located at a given height of the subject 30.

Concurrently with the processing for reconstructing the first X-ray tomographic image, the second image correction unit 42 performs a correction for correcting the identified abnormal pixel data corresponding to the defective pixels; the second image reconstruction unit 46 uses the projection data of the X-ray images corrected by the second image correction unit 42 to reconstruct the second X-ray tomographic image at the cross section located at the same height of the subject 30 as does the first image reconstruction unit 44.

The selector 48 switches between the first X-ray tomographic image and the second X-ray tomographic image at a given timing according to the selection signal to output the selected X-ray tomographic image.

The X-ray tomographic image outputted from the selector 48 is displayed on the monitor 20. Controlled by the controller 14, the monitor 20 indicates information on the display status of the X-ray tomographic image (information showing which of the first and second X-ray tomographic images is displayed) and also displays an input screen for entering an instruction using the input unit 12 for selectively displaying the first X-ray tomographic image and the second X-ray tomographic image.

The user of the imaging system 10 can know by referring to the X-ray tomographic image display status information whether the X-ray tomographic image displayed on the monitor 20 is the first X-ray tomographic image or the second X-ray tomographic image. The user can freely switch between the first X-ray tomographic image and the second X-ray tomographic image at any timing desired by issuing a switching instruction through the instruction input screen from the input unit 12.

The X-ray tomographic image outputted from the selector 48 is supplied to the output unit 22, which, for example, prints out the X-ray tomographic image and allows digital image data of the X-ray tomographic image to be stored in a recording medium.

The imaging system 10 uses not the abnormal pixel data corresponding to the defective pixels but only the pixel data corresponding to the normal pixels to perform image processing and thereby reconstructs the X-ray tomographic image at a cross section located at a given height of the subject 30. Therefore the first X-ray tomographic image can be displayed at a higher speed in a shorter time period. A switch may also be made from the first X-ray tomographic image to the higher-definition second X-ray tomographic image. Alternatively, a switch may be made between the two images at any timing.

The specific structure of each component of the X-ray imaging system of the invention is not limited in any manner and may be achieved by any of various means used to provide similar functions. The correction made by the first image correction unit 40 is not limited to masking so long as the X-ray tomographic image can be reconstructed in the first image reconstruction unit 44 not using the abnormal pixel data corresponding to the defective pixels but using only the pixel data corresponding to the normal pixels.

The present invention is basically as described above.

The present invention, described above in detail, is not limited in any manner to the above embodiments and various improvements and modifications may be made without departing from the spirit of the invention.

We claim:

1. An X-ray imaging system comprising:
an imager in which a subject is irradiated with X-ray at different angles while moving an X-ray source in one direction and the X-ray with which said subject has been irradiated is detected with a flat panel detector to acquire projection data of X-ray images taken at the different angles in tomosynthesis imaging; and
an image processor in which an X-ray tomographic image is reconstructed not by using abnormal pixel data corresponding to defective pixels but by using only pixel data corresponding to normal pixels from among pixel data making up the projection data of the X-ray images acquired by said imager by reference to a defective pixel map in which information on the defective pixels due to said flat panel detector and its peripheral circuit has been stored.

2. The X-ray imaging system according to claim 1, wherein said image processor comprises:
an abnormal pixel data identification unit which identifies the abnormal pixel data corresponding to the defective pixels due to said flat panel detector and its peripheral circuit from among the pixel data making up the projection data of the X-ray images acquired by said imager by reference to said defective pixel map;
a first image correction unit which performs a correction for masking the abnormal pixel data corresponding to the defective pixels identified by said abnormal pixel data identification unit; and
a first image reconstruction unit which uses the projection data of the X-ray images corrected by said first correction unit to reconstruct a first X-ray tomographic image.

3. The X-ray imaging system according to claim 2, wherein said image processor further comprises:
a second image correction unit which corrects each of the abnormal pixel data corresponding to the defective pixels identified by said abnormal pixel data identification unit for each of said X-ray images acquired by said imager;
a second image reconstruction unit which uses the projection data of the X-ray images corrected by said second image correction unit to reconstruct a second X-ray tomographic image; and
a selector which switches between said first X-ray tomographic image reconstructed by said first image reconstruction unit and said second X-ray tomographic image reconstructed by said second image reconstruction unit at a given timing to selectively output said first X-ray tomographic image or said second X-ray tomographic image.

4. The X-ray imaging system according to claim 3, wherein said selector outputs said first X-ray tomographic image after said first image reconstruction unit has completed reconstruction of said first X-ray tomographic image and outputs said second X-ray tomographic image after said second image reconstruction unit has completed reconstruction of said second X-ray tomographic image.

5. The X-ray imaging system according to claim 4, wherein after a predetermined time period has elapsed from completion of the reconstruction of said second X-ray tomographic image, said selector switches from said first X-ray tomographic image to said second X-ray tomographic image and outputs said second X-ray tomographic image.

6. The X-ray imaging system according to claim 3, wherein after reconstruction of said second X-ray tomographic image has been completed, said selector selectively outputs said first X-ray tomographic image or said second X-ray tomographic image according to an instruction entered from an outside.

7. The X-ray imaging system according to claim 3, wherein after said first image reconstruction unit has completed reconstruction of said first X-ray tomographic image, said selector outputs said first X-ray tomographic image and for portions in which the abnormal pixel data was not used to reconstruct said first X-ray tomographic image, abnormal pixel data portions in said first X-ray tomographic image are sequentially replaced by their corresponding abnormal pixel data portions in the reconstructed second X-ray tomographic image each time one abnormal pixel data portion of said second X-ray tomographic image is reconstructed in said second image reconstruction unit until reconstruction of said second X-ray tomographic image is completed.

8. The X-ray imaging system according to claim 3, further comprising:
an input unit for entering various instructions;
a controller for controlling operations of said X-ray imaging system according to an instruction entered from said input unit; and
a monitor for displaying said X-ray tomographic image outputted from said selector,
wherein said controller causes said monitor to indicate information on display status as to whether said first X-ray tomographic image is displayed or said second X-ray tomographic image is displayed.

9. The X-ray imaging system according to claim 8, wherein said controller causes said monitor to indicate an input screen for entering an instruction from said input unit for selectively displaying said first X-ray tomographic image or said second X-ray tomographic image.

10. The X-ray imaging system according to claim 3, wherein said second image reconstruction unit uses the abnormal pixel data corresponding to the defective pixels identified by said abnormal pixel data identification unit to reconstruct only pixels of said second X-ray tomographic image corresponding to said defective pixels.

11. The X-ray imaging system according to claim 3, wherein said second image correction unit uses pixel data of normal pixels surrounding each of abnormal pixels on an identical X-ray image to correct the abnormal pixel data corresponding to the defective pixels.

12. The X-ray imaging system according to claim 3, wherein said second image correction unit utilizes projection data of other X-ray images acquired by taking at a different angle, and uses pixel data of normal pixels of said other X-ray images corresponding to abnormal pixels and surrounding normal pixels to correct the abnormal pixel data corresponding to the defective pixels.

13. An X-ray imaging method comprising the steps of:
irradiating a subject with X-ray at different angles while moving an X-ray source in one direction, and detecting the X-ray with which said subject has been irradiated with a flat panel detector to acquire projection data of X-ray images taken at the different angles in tomosynthesis imaging; and
reconstructing an X-ray tomographic image not by using abnormal pixel data corresponding to defective pixels but by using only pixel data corresponding to normal pixels from among pixel data making up the projection data of the X-ray images by reference to a defective pixel map in which information on the defective pixels due to said flat panel detector and its peripheral circuit has been stored.

14. The X-ray imaging method according to claim 13, wherein reconstruction of said X-ray tomographic image comprises:
identifying the abnormal pixel data corresponding to the defective pixels of said flat panel detector from among the pixel data making up the acquired projection data of the X-ray images by reference to said defective pixel map;
performing a first correction for masking the identified abnormal pixel data corresponding to the defective pixels; and
reconstructing a first X-ray tomographic image by using the projection data of the X-ray images after said first correction.

15. The X-ray imaging method according to claim 14, further comprising:
performing a second correction for correcting the identified abnormal pixel data corresponding to said defective pixels;
reconstructing a second X-ray tomographic image by using the projection data of the X-ray images after said second correction; and
selectively outputting said first X-ray tomographic image or said second X-ray tomographic image at a given timing.

16. A computer readable media including an X-ray imaging program for causing a computer to execute:
a step of receiving projection data of X-ray images taken at different angles acquired by irradiating a subject with X-ray at the different angles while moving an X-ray source in one direction and detecting the X-ray with which said subject has been irradiated with a flat panel detector in tomosynthesis imaging;
a step of identifying abnormal pixel data corresponding to defective pixels of said flat panel detector from among pixel data making up the received projection data of the X-ray images by reference to a defective pixel map in which information on the defective pixels due to said flat panel detector and its peripheral circuit has been stored;
a step of performing a first correction for masking the identified abnormal pixel data corresponding to the defective pixel; and
a step of reconstructing a first X-ray tomographic image by using the projection data of the X-ray images after said first correction.

17. The computer readable media including the X-ray imaging program according to claim 16 for causing the computer to further execute:
a step of performing a second correction for correcting the identified abnormal pixel data corresponding to the defective pixels;
a step of reconstructing a second X-ray tomographic image by using the projection data of the X-ray images after said second correction; and
selectively outputting said first X-ray tomographic image or said second X-ray tomographic image at a given timing.
